(19) United States
(12) Patent Application Publication
(10) Pub. No.: US 2026/0277421 A1
(43) Pub. Date: Sep. 17, 2026

(54) SYSTEM AND METHOD FOR PERFORMING A CHEMICAL SUSTAINABILITY ASSESSMENT

(71) Applicant: THE BOEING COMPANY, Arlington, VA (US)

(72) Inventors: Shirell Eliezra Klein, Agoura Hills, CA (US); Ashley Marie Dustin, Santa Monica, CA (US); Melinda Dae Miller, Snohomish, WA (US); Jill E Seebergh, Seattle, WA (US); Josi May Herron, Seattle, WA (US); Kajsa Magdalena Williams, Malibu, CA (US)

(73) Assignee: THE BOEING COMPANY, Arlington, VA (US)

(21) Appl. No.: 19/545,266

(22) Filed: Feb. 20, 2026

Related U.S. Application Data
(60) Provisional application No. 63/770,683, filed on Mar. 12, 2025.

(51) Int. Cl. G06F 3/04847; G06V 30/148

(52) U.S. Cl. CPC G06F 3/04847; G06V 30/153

(57) ABSTRACT

A chemical sustainability assessment system and method include receiving a first input from a user via a software application operating on a computing device, and identifying a chemical and composition information for the chemical based on the first input. The method controls a display device to display a graphical user interface (GUI) that identifies sustainability parameters and presents weight settings for the sustainability parameters. The method receives a second input from the user adjusting the weight setting associated with at least one of the sustainability parameters to provide user-adjusted weight settings. The method accesses multiple data sources to generate scores for at least some of the sustainability parameters, and generates a sustainability value for the chemical based on the scores that are generated and the user-adjusted weight settings. The method controls the display device to display the sustainability value on the GUI.

### CROSS REFERENCE TO RELATED APPLICATION

This application relates to and claims priority benefits from U.S. Provisional Patent Application No. 63/770,683, filed Mar. 12, 2025, which is hereby incorporated by reference in its entirety.

### FIELD

The subject matter herein relates to an interactive system and method for assessing the sustainability of chemicals.

### BACKGROUND

A chemical sustainability assessment tool operates on mobile applications or devices to provide sustainability assessment of chemical substances and mixtures. Chemical sustainability assessment tools, such as life cycle analysis (LCA) calculators, provide insights for evaluating chemicals and chemical mixtures and facilitate identifying chemicals that are more sustainable and/or safer for human health and the environment than other chemicals. Known chemical sustainability assessment tools have several drawbacks that limit the effectiveness of these tools. For example, some known tools require the user to input extensive information about the test chemical which makes the tools complex and difficult to use. Furthermore, some tools may not provide clear comparison metrics, so it is difficult for the user to determine the basis for a given score or comparison between two or more chemicals. In addition, some known tools are limited to a small sample of sustainability factors or parameters, and do not consider other relevant sustainability parameters. As an example, some tools may include assessments of one or two parameters, such as toxicology and/or atmospheric effects, but do not include other endpoints that affect both sustainability and feasibility of use and would be relevant for evaluating a use application of the chemical. These other endpoints or parameters that may not be considered in existing chemical sustainability assessment tools can include flammability, reactivity, volatile organic compound (VOC) content, hazardous waste production, regulatory risk, cost, and the like. Due to the limited scope, the existing tools do not provide a broad, holistic approach for assessing the test chemicals.

Furthermore, the known chemical sustainability assessment tools do not permit the user to select and adjust the importance of the different sustainability parameters that are evaluated to generate an assessment value (e.g., score) for the test chemical. For example, the known tools are not user tunable, meaning that the tools lack scaling controls that are user adjustable. This deficiency limits the effectiveness of the known tools for evaluating test chemicals for different potential use cases/applications of the test chemicals. For example, a first sustainability parameter may be more relevant than (e.g., prioritized over) a second sustainability parameter for a particular technological application, but the first and second sustainability parameters may be factored equally in the chemical assessment by one or more of the known chemical sustainability assessment tools. The user may not be able to scale the importance of the sustainability parameters to focus on different intended use applications.

### SUMMARY

In one or more embodiments, a computer-implemented method for performing a chemical sustainability assessment is provided. The method includes receiving a first input from a user via a software application operating on a computing device, and identifying, via one or more processors of the computing device, a chemical and composition information for the chemical based on the first input. The method includes controlling a display device to display a graphical user interface (GUI) that identifies sustainability parameters and presents weight settings for the sustainability parameters. A second input is received from the user via the software application. The second input adjusts the weight setting associated with at least one of the sustainability parameters to provide user-adjusted weight settings. The method includes accessing multiple data sources to generate scores for at least some of the sustainability parameters. The method includes generating a sustainability value for the chemical based on the scores that are generated and the user-adjusted weight settings, and controlling the display device to display the sustainability value for the chemical on the GUI.

In one or more embodiments, a system for performing a chemical sustainability assessment is provided. The system includes one or more memory devices, that are non-transitory and computer-readable, and one or more processors of a computing device operably connected to the one or more memory devices. The one or more processors are configured to receive a first input from a user via a software application operating on the computing device, and identify a chemical and composition information for the chemical based on the first input. The one or more processors are configured to control a display device to display a GUI that identifies sustainability parameters and presents weight settings for the sustainability parameters. The one or more processors are configured to receive a second input from the user via the software application. The second input adjusts the weight setting associated with at least one of the sustainability parameters to provide user-adjusted weight settings. The one or more processors are configured to access multiple data sources to generate scores for at least some of the sustainability parameters. The one or more processors are configured to generate a sustainability value for the chemical based on the scores that are generated and the user-adjusted weight settings, and control the display device to display the sustainability value for the chemical on the GUI.

### BRIEF DESCRIPTION OF THE DRAWINGS

FIG. 1 is a block diagram illustrating a chemical sustainability assessment (CSA) system formed in accordance with embodiments.

FIG. 2 illustrates a computing device of the CSA system according to an embodiment.

FIG. 3A is a diagram showing an example workflow of the CSA system according to an embodiment.

FIG. 3B is a continuation of the diagram showing the example workflow of the CSA system of FIG. 3A according to an embodiment.

FIG. 4A shows an example assessment setup page on a graphical user interface (GUI) of the CSA system according to an embodiment.

FIG. 4B shows a continuation of the example assessment setup page on a graphical user interface (GUI) of the CSA system of FIG. 4A according to an embodiment.

FIG. 5A shows an example assessment result page on the GUI of the CSA system according to an embodiment.

FIG. 5B shows a continuation of the example assessment result page on the GUI of the CSA system of FIG. 5A according to an embodiment.

FIG. 5C shows a continuation of the example assessment result page on the GUI of the CSA system of FIG. 5B according to an embodiment.

FIG. 5D shows a continuation of the example assessment result page on the GUI of the CSA system of FIG. 5C according to an embodiment.

FIG. 6A shows an example comparison page on the GUI of the CSA system according to an embodiment.

FIG. 6B shows a continuation of the example comparison page on the GUI of the CSA system of FIG. 6A according to an embodiment.

FIG. 7 is a flow chart of a method for performing a sustainability assessment for a chemical according to an embodiment.

### DETAILED DESCRIPTION

Embodiments described herein are directed to a chemical sustainability assessment system and method that dynamically perform sustainability assessments of chemicals on a multitude of sustainability parameters to generate a holistic (e.g., totality of the circumstances) metric for each evaluated chemical. The metric may be a quantitative value that enables simple comparisons between multiple different chemicals or chemical mixtures. The sustainability parameters are factors or endpoints which represent different categories in which the test chemical can be evaluated. The chemical sustainability assessment (CSA) system can incorporate sustainability parameters related to environmental, health, manufacturability, regulatory, and feasibility factors. In an example, the CSA system may evaluate sustainability parameters, at a chemistry level, including toxic use reduction, reactivity, flammability, global warming potential (GWP), volatile organic compound (VOC) content, ozone depletion, regulatory risk, hazardous waste disposal, and/or cost. The CSA system provides insights for evaluating the test chemical, such as how environmentally friendly or “green” the chemical is, what kind of regulatory risk would be applicable by using the chemical, how would the chemical potentially affect (e.g., contribute to) global warming if selected for use, what is the flammability of the chemical, what is the reactivity of the chemical, and/or the like. The CSA system provides these insights regarding the sustainability parameters to allow the user to make informed chemical selections in consideration of environment impact.

The CSA system may operate at least in part on a computing device that is accessible to a user. For example, the CSA system may be a program that displays a graphical user interface (GUI) on a display screen. The computing device may be a mobile computing device, such as a smart phone or tablet computer. In another example, the computing device may be a desktop computer, workstation, laptop computer, or the like. The user may interact with the CSA system via one or more user input devices connected to the computing device. For example, the user may input information, via the user input device, that identifies a specific chemical (e.g., a test chemical) to evaluate by running a chemical sustainability assessment. The GUI may display information about the evaluated chemical for observation by the user. The displayed information may include identification information, regulatory information, warning information, an assessment value (e.g., score), and/or the like. The CSA system may dynamically update the GUI to accommodate and incorporate additional user inputs and user-based scaling of the importance (e.g., weights) of the sustainability parameters in the assessment.

The CSA system can also be used to directly compare two or more chemicals. For example, a user may desire a comparison between a first, incumbent chemical that is currently used in a formulation or product and a second, candidate chemical being considered as a replacement for the first chemical in the formulation or product. As an example, the incumbent chemical may be identified in a regulation as a chemical to phase out of use in a designated time horizon (e.g., 5 or 10 years). The comparison function of the CSA system described herein may assist with determining a replacement chemical to be used in place of the incumbent chemical. As another example, the comparison function can be used for marketing purposes, such as to tout that a new chemical or formulation is more sustainable and/or environmentally friendly than an existing chemical already in use. The CSA system can assist the user with selecting chemicals for various different use applications (e.g., products that incorporate the chemicals). Some example use applications or products include paints, sealants, cleaners, and the like. The CSA system and method provides rapid assessments to guide sustainable replacements and early-stage product development with the added ability to control the scope of analysis.

One or more technical effects of the CSA system and method described herein including providing a holistic approach that considers a broad swath of sustainability factors from a use application perspective. The CSA system may consider a broader range of parameters than known sustainability tools, including parameters that are not typically present in the known sustainability tools. For example, the CSA system may evaluate the flammability, reactivity, VOC, hazardous waste production, regulatory risk, and cost of the chemical. The CSA system may account for human health hazards, ecological hazards, atmospheric hazards, industrial production requirements, regulatory risk, and economic feasibility. The breadth of this assessment quantifies sustainability from a holistic business case perspective, highlighting sustainable advantages and disadvantages associated with the chemical. The CSA system in an example may also factor cost into the assessment value.

Another technical effect of the CSA system and method includes enabling dynamic user scaling (e.g., tuning) of the sustainability parameters, which affects the quantitative assessment value that is output. The CSA system and method enables the user to set the importance of each of the sustainability parameters by adjusting scaling factors. The user can therefore customize the assessment based on different intended use applications. The CSA system can provide application-driven comparisons, because intended technological applications may drive prioritization and emphasis of certain sustainability parameters over other sustainability parameters. As a result, the CSA system is outputs a quantifiable, application-driven assessment value that is easily compared to other chemicals. While other tools have a standard calculation algorithm, the CSA system can be tuned in real-time by the user to reflect the projected application goals and/or needs. Rather than requiring multiple tools, calculators, and/or subject matter expertise to capture the most important endpoints, the CSA system described herein readily adapts to individual use cases.

Another technical effect is that the CSA system and method is relatively simple and easy to use for the user, requiring only a limited amount of information from the user. For example, the user may simply launch the CSA program on the computing device, and then select, upload, or otherwise type in an identifier of the chemical before running the assessment. The user may only need to identify the constituent components of the chemical and the relative proportions (e.g., weight percentages) of the constituent components. In a first example, the CSA system may only require the user to provide a chemical ID entry and % composition. In a second example, the user may simply upload or select a safety data sheet (SDS) that identifies the chemical to assess. The CSA system may obtain all other data at the back end server side. The CSA system may be more user-friendly than other sustainability tools that require the user to know the exact emissions produced, toxicological effects, physical properties, and/or the like. When using other sustainability tools, the user may also need to know the data sources to find this information. The CSA system is integrated with a variety of data sources to automatically incorporates information from various sources.

The CSA system beneficially outputs a quantitative assessment value for the evaluated chemical that allows for clear comparisons. For example, the assessment value may be a numeric value, such as an integer, a number with one or more decimal places, or the like. Some other sustainability tools have broad, qualitative outputs, such as alphabetic scoring (e.g., A, B, C, D, F, etc.) or good/neutral/bad, which makes comparing chemicals difficult. With reference to the user-scaling of the importance of the sustainability parameters, the CSA system and method may automatically use consistent weight settings of the sustainability parameters when comparing chemicals to provide a reliable comparison.

As an example, the CSA system and method described herein may promote sustainable technology development. The CSA system and method may perform rapid, broad, and tunable quantitative sustainability assessments to guide research and development efforts. The CSA system may be employed for various applications, including trade studies, chemistry downselection, vetting technology for regulatory concerns at project inception, and identifying green replacements for chemicals in existing products.

The CSA system and method demonstrate an improvement in the basic ability of a computing device to interact with users and provides a technical solution to a technical problem. The computing device on which the CSA system operates is better able to interact with users by permitting users to dynamically adjust the prominence of the sustainability parameters relative to one another. The weight settings of the sustainability parameters have a direct influence on the assessment value that is generated by the CSA system and used to compare the evaluated chemical to other chemicals. The CSA system also represents a specific technical improvement over prior sustainability tools and calculators that cannot adjust the relative importance (e.g., the weight settings) of the sustainability parameters in the assessment. The problem of failure to provide adjustable weight settings for the sustainability parameters is necessarily rooted in computer technology. The known sustainability tools and calculators are not able to adjust the calculations by increasing or decreasing the prominence of some sustainability parameters relative to other sustainability parameters. The known tools and calculators also do not permit a user to adjust the calculation by adjusting the weights of the sustainability parameters. The CSA system and method also provide an improved user interface application for mobile computing devices. These and other technical benefits of the CSA system and method are described in more detail herein.

The terms “user” and “users” refer to persons that operate the computing device that runs the CSA system and method to perform the sustainability assessment. The terms “test chemical” and “chemical” refer to a chemical substance or material that is assessed by the CSA system and method. The test chemical and chemical may be a pure chemical compound (e.g., substance) or a mixture of multiple chemical compounds. In the case of a mixture, the different compounds or substances in the mixture are referred to herein as “components” and “constituent components.”

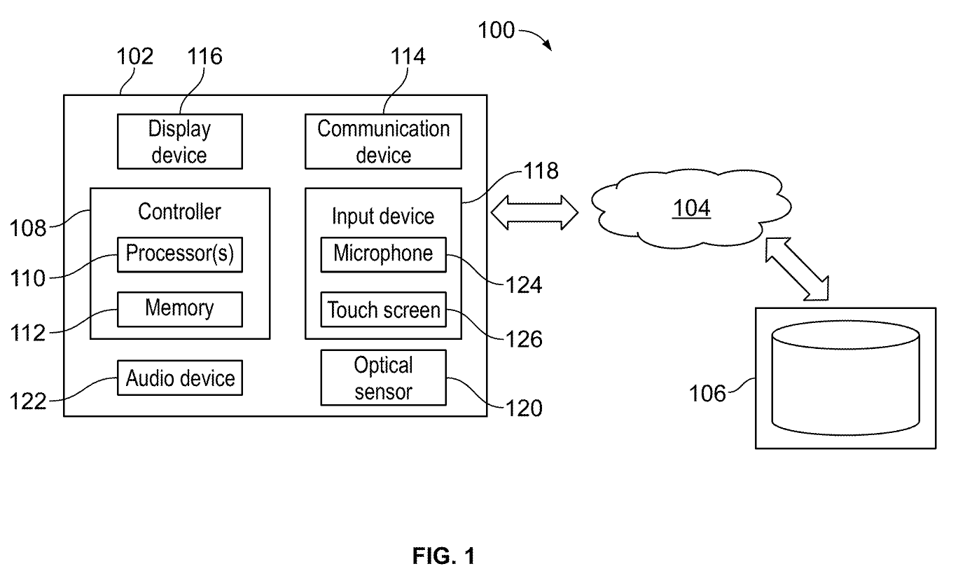

FIG. 1 is a block diagram illustrating a chemical sustainability assessment (CSA) system 100 formed in accordance with embodiments herein. The CSA system 100 includes a computing device 102 that is capable of communicating via a network 104 with one or more remote computing devices 106. The remote computing device(s) 106 may include one or more servers, computer towers, desktop computers, workstations, and/or the like. The remote computing device(s) 106 may be located at a remote computing facility, such as a cloud computing facility. The computing device 102 is operated by a user. The computing device 102 may be a mobile or personal computing device, such as a phone (e.g., smartphone), tablet computer, laptop computer, or the like. In some examples, the computing device 102 may be portable and/or handheld. The computing device 102 performs at least some of the operations described herein to perform a sustainability assessment of a chemical and compare multiple chemicals.

In an example, the computing device 102 and the remote computing device(s) 106 may form a cloud-type computing system (e.g., public cloud, private cloud, or hybrid cloud). The network 104 may be a cable network, the Internet, a private intranet, a local area network, a wide area network, a wireless network, and/or the like. The communication pathways of the network 104 between the computing device 102 and the remote computing device(s) 106 may be defined by wireless segments and/or wired segments. For example, the computing device 102 may wirelessly communicate with one or more cell towers, and the cell towers may communicate through a wired connection to the remote computing device 106 at a remote cloud storage facility to complete the communication pathway.

The computing device 102 includes a controller 108. The controller 108 represents hardware circuitry that includes and/or is connected with one or more processors 110 (e.g., one or more microprocessors, integrated circuits, microcontrollers, field programmable gate arrays, etc.). The controller 108 may include one or more application servers and, optionally, one or more web servers. The controller 108 includes and/or is connected with at least one tangible and non-transitory computer-readable storage medium 112 (referred to herein as memory and memory device). The memory 112 may be or include a random-access memory, a flash memory, a magnetic/optical storage, or the like. The processor(s) 110 are communicatively connected to the at least one memory 112, such as via electrical wires, electrical cables, circuit traces, fiber optics, and/or the like.

The memory 112 may store computer-readable programmed instructions (e.g., software) that are executed by the processor(s) 110 to perform at least some of the operations described herein. The programmed instructions can instruct the processor(s) 110 how to implement a software application on the computing device 102 to perform a sustainability assessment of a chemical and compare multiple chemicals. For example, the programmed instructions may instruct the processor(s) 110 how to generate a graphical user interface (GUI) for the software application that is displayed on a display device. The memory 112 may be used as a repository for storing various information accessed and utilized by the processor(s) 110 when operating the software application. For example, the memory 112 (or another memory device of the computing device 102) may store user information about previous chemical assessments performed by the CSA system 100, different specific users (e.g., user accounts), one or more algorithms for generating the assessment value for a test chemical, one or more algorithms for scaling the weight settings of the sustainability parameters, and/or the like. The processor(s) 110 may access various portions of the memory 112 to obtain (e.g., retrieve) relevant information for performing the sustainability assessment and presenting the relevant information and interactivity to enhance the user experience.

The computing device 102 may have a communication device 114 that enables the controller 108 to send and receive information via the network 104. The communication device 114 can represent circuitry that can communicate electrical signals wirelessly and/or via wired connections. For example, the communication device 114 can include transceiving circuitry, one or more antennas, modems, routers, or the like. The transceiving circuitry may include a transceiver or separate transmitter and receiver devices. The electrical signals can form data packets that in the aggregate represent messages, such as request messages and response messages. In various embodiments, the controller 108 may use the communication device 114 to communicate with the one or more remote computing devices 106 via the network 104. The controller 108 may communicate with the remote computing devices 106 to implement and perform the operations of the CSA system and method described herein. The controller 108 may generate messages that are remotely communicated by the communication device 114 to the remote computing devices 106. The controller 108 may receive messages, from the remote computing devices 106, that are initially received by the communication device 114 and forwarded to the controller 108.

The computing device 102 may include or may be communicatively connected to a display device 116. The display device 116 is controlled by the controller 108 to display visual content to the user on a screen of the display device 116. The visual content includes graphical indicia, such as images, shapes, text, and symbols. The display device 116 may present a graphical user interface (GUI) of a software application or website that implements the CSA system 100 and method described herein. The display device 116 may be integrated into the computing device 102, such as within the housing of the mobile computing device 102. Alternatively, the display device 116 may be a separate monitor that is communicatively connected to the computing device 102 via a wired or wireless connection.

The computing device 102 may include at least one input device 118 (also referred to herein as user input device). Each input device 118 is operable to enable the user of the computing device 102 to interact with the CSA system 100. The user may actuate the input device(s) 118 to submit user commands and/or selections for providing information to and/or controlling operation of the CSA system 100. The input device 118 generates a control signal, in response to the user’s input, which is communicated to the controller 108. The input device(s) 118 may include a touch sensitive display screen 126, a touch pad, a mouse, a keyboard, a microphone 124, a joystick, physical buttons, and/or the like. For example, the user may use one of the input devices 118 to download or launch a software application or program of the CSA system 100 and to interact with the CSA system 100. The user may interact with the CSA system 100 by inputting information to identify a chemical to evaluate, adjusting the weight settings of one or more of the sustainability parameters, triggering the CSA system 100 to begin or “run” an assessment, and/or the like. In an example, the computing device 102 may include multiple user input devices 118, such as a microphone 124 for receiving user voice commands and a touch screen 126, touch pad, and/or keyboard for receiving user touch inputs.

Optionally, the computing device 102 may include at least one optical sensor 120. The optical sensor 120 may be used by the user to identify a chemical to assess. The user can upload imaged or scanned data into the CSA system 100, which is analyzed by the controller 108, to avoid the user having to manually type the input information. The optical sensor 120 may include a camera, an electromagnetic scanner or reader device, and/or the like. For example, the optical sensor 120 may be a camera that captures (e.g., generates) image data based on light in the surrounding environment that enters a lens and impinges upon a digital sensor of the camera. The user may photograph an SDS using the camera to identify a chemical to assess. For example, the controller 108 may perform optical character recognition on the image data generated by the camera to determine the printed content on the SDS. In another example, the optical sensor 120 may be an electromagnetic scanner or reader that scans a code printed on a tangible item, such as an SDS or the like. The code may be a barcode, a quick-response (QR) code, or a radio frequency identification (RFID) tag. The controller 108 may identify one or more chemicals based on the scanned code. The controller 108 may automatically populate input boxes of the GUI of the CSA system 100 based on the information parsed from the data collected by the optical sensor 120. The user can modify the populated information in the input boxes as desired using an input device 118.

Optionally, the computing device 102 may include at least one audio device 122. The audio device(s) 122 may be a speaker with an amplifier to emit sounds based on electrical signals. The controller 108 may generate the electrical signals that are communicated to the audio device(s) 122 and output as audio. As an example, the controller 108 may control the audio device(s) 122 to emit machine-generated speech and/or human-recorded speech. The audible speech that is emitted from the audio device(s) 122 may provide information about the chemical sustainability assessment to the user, such as the assessment value that is generated for the chemical that is evaluated.

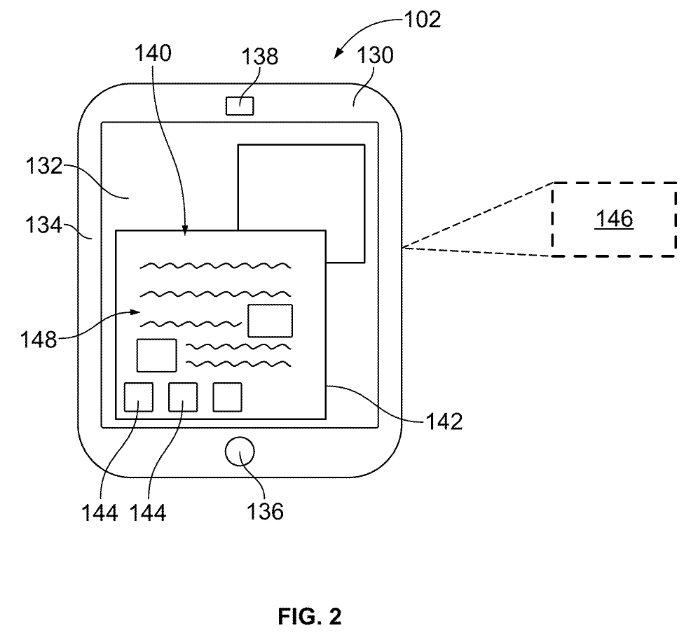

FIG. 2 illustrates the computing device 102 of the CSA system 100 according to an embodiment. The computing device 102 in FIG. 2 is illustrated as a tablet computer. For example, the computing device 102 includes a housing 130 that is relatively flat and planar. A display screen 132 of the display device 116 (shown in FIG. 1) occupies a majority of the front, or user-facing, side 134 of the computing device 102. The housing 130 may frame the display screen 132. The display screen 132 may be touch-sensitive, to also function as an input device 118 (shown in FIG. 1). The computing device 102 may also have additional input devices 118, such as a user-selectable button 136 next to the display screen 132. The mobile computing device 102 may include a camera 138 that represents an optical sensor 120 (shown in FIG. 1). The user may employ the camera 138 for optically scanning or imaging an SDS or other document for identifying a chemical to evaluate. The tablet-style computing device 102 shown in FIG. 2 is merely an example computing device on which the CSA system 100 could be implemented. The computing device 102 may have other form factors, shapes, sizes, and the like in other examples. For example, the CSA system 100 could be implemented on various different types of computing devices, including other types of mobile computing devices 102.

The controller 108 (shown in FIG. 1) of the mobile computing device 102 may control the display device 116 to present a graphical user interface (GUI) 140 for the CSA system 100 on the display screen 132. The GUI 140 manages the interaction between the CSA system 100 and the user through graphical elements, such as windows 142 and user-selectable input elements 144. The windows 142 display various types of information for various computer processes corresponding to the CSA system 100. Multiple windows 142 may be concurrently displayed. The GUI 140 may present a pop-up window 148 that notifies the user of important regulatory information and/or data acquisition warnings. The user can selectively close the pop-up window 148 to view the main assessment page of the GUI 140. The GUI 140 140 may include images, symbols, text, illustrations, videos, and/or the like.

The user-selectable input elements 144 (referred to herein as input elements) accept user input for controlling the computer processes of the CSA system 100. The input elements 144 may be virtual buttons that are selectable via a touch input on the area of the display screen 132 showing the input element 144 or via a user-movable cursor that is displayed on the screen 132. In an example, a first input element 144 may be selectable by the user to initiate or run a sustainability assessment once a chemical is identified to evaluate and the controller 108 has sufficient information to run the assessment. A second input element 144 may be selectable by the user to enable the user to adjust the weight settings of one or more of the sustainability parameters. A third input element 144 may be selectable to enable the user to submit an inquiry or command, such as a voice command. The GUI 140 may have any number of input elements 144 for allowing the user to interact with the CSA system 100.

In an embodiment, the CSA system 100 may be voice compatible to enable a user to select desired input elements 144 via voice commands, without requiring touch inputs. For example, a user may press a button or state out loud a trigger term to activate the microphone 124. With the microphone 124 active, the user may then say one of the input elements 144 to select that corresponding input element 144.

The CSA system 100 may include a software application 146 that operates on the computing device 102. The software application 146 is a program that may be stored locally on the mobile computing device 102, such as in the memory 112 or another local non-transitory computer-readable storage medium. The software application 146 permits the user to interact with the CSA system 100. For example, the software application 146 may generate the GUI 140 that displays graphical content to the user and receives user input. The controller 108 may host the software application 146, including the tools, application program interfaces (APIs), and scripts, among other things, that may be used for the software application 146. Optionally, a user may download the software application 146 for the CSA system 100 onto their computing device 102. The user may open, launch, or initiate the software application 146 to access and implement the CSA system 100. As an example, the user may open the software application 146 by selecting an icon that represents the software application 146. The software application 146 may require at least one form of authentication from the user before granting the user access to a personal account. Accepted forms of authorization may include a password, a security code on a trusted device, face or other biometric recognition, and/or the like.

Optionally, the software application 146 may be configured to work with sub-applications or scripts (e.g., plug-ins or extensions) that are executed from within the software application 146 or in concert with the software application 146. The software application 146 may retrieve and communicate information through the network 104 (shown in FIG. 1), such as by communicating with the remote computing device(s) 106. The software application 146 may communicate using, for example, a known protocol (e.g., Hypertext Transfer Protocol (HTTP) or HTTP-secure (HTTPS)). More specifically, the software application 146 may send requests (e.g., HTTP requests) for information to any web-accessible internet address. The software application 146 may display information on the computing device 102 in accordance with a predetermined format (e.g., HTML format). Embodiments set forth herein may be implemented, at least in part, using the software application 146, a sub-application associated with the software application 146, or other software program having computer executable code.

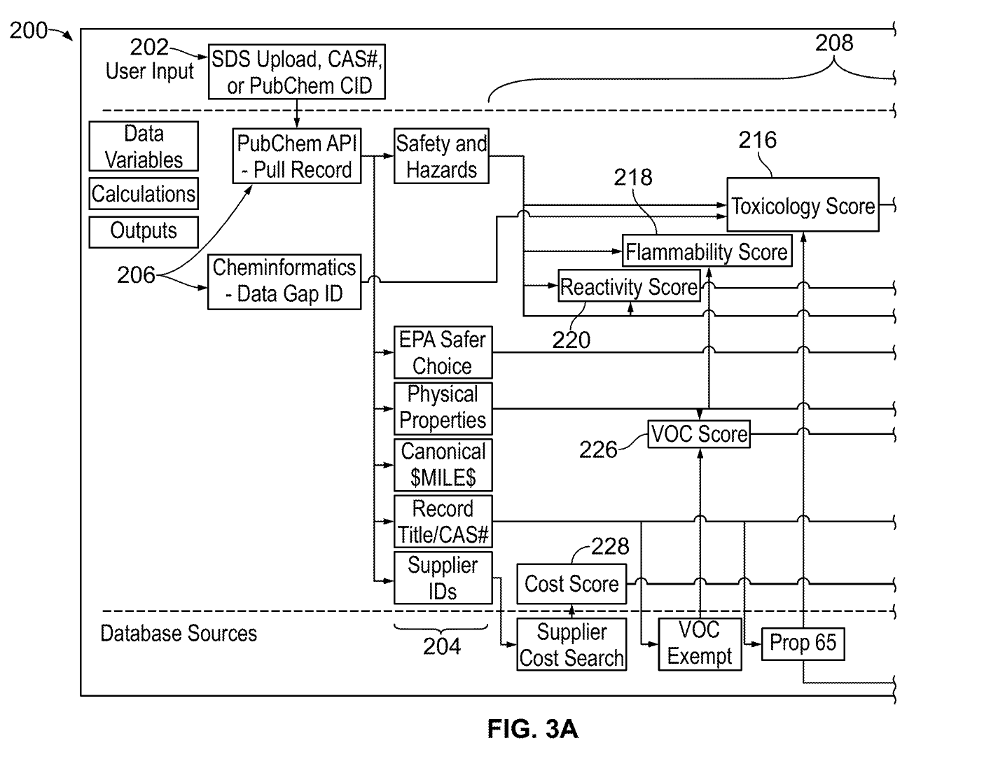

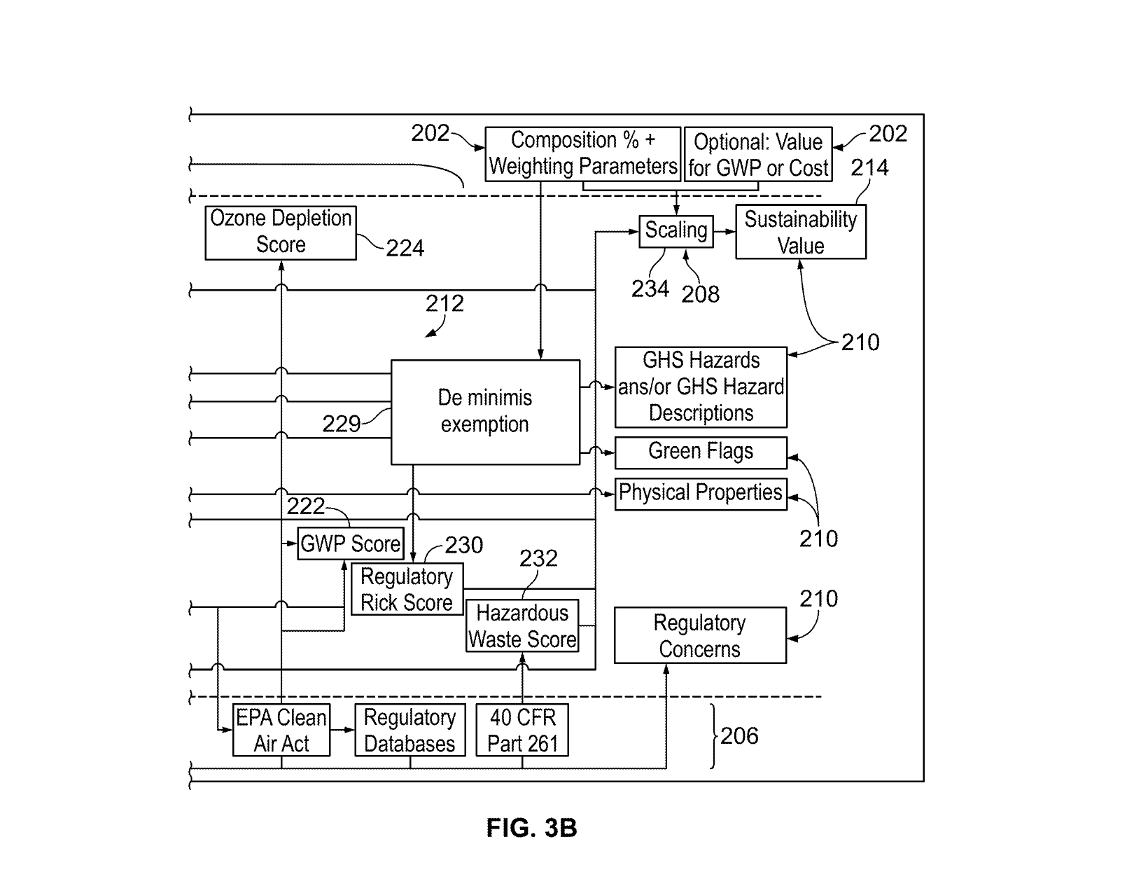

FIG. 3 is a diagram 200 showing an example workflow of the CSA system 100 according to an embodiment. The workflow shows various user inputs 202, data variables 204, database searches 206, calculation steps 208, and generated outputs 210. The information presented in the diagram 200 is provided to describe an example formulation of the CSA system 100, and is not intended to represent all embodiments. For example, the data variables 204, database sources 206, calculation steps 208, and outputs 210 are exemplary only. The calculation steps 208 may be performed by the controller 108 of the CSA system 100.

To run an assessment, the user may launch the CSA system 100 program and then log in by providing user credentials. The user may log in to an existing user account or sign up for a new account. Each existing user account may be associated with a user identifier that is stored in a memory device. This allows persistent access to started assessments. Once viewing an assessment page of the GUI 140, the user may provide a first input via the application. The first input provides composition information for the chemical to be evaluated. The composition information identifies the one or more constituent components of the chemical and a relative percentage of each of the components. For a mixture that includes equal parts of four compounds A, B, C, and D, the composition information in the first input may identify 25 wt.% A, 25 wt.% B, 25 wt.% C, and 25 wt.% D. The CSA system 100 may require that the percentages add to 100.

The CSA system 100 enhances ease of use by offering the user different means to provide the first input that identifies the chemical to be evaluated. For example, the user can manually type in the composition information into corresponding boxes displayed on the GUI 140, via the touch screen 126 or another input device 118. Alternatively, or in addition, the CSA system 100 enables the user to enter a PubChem Compound Identification (PubChem CID) for the chemical and/or CAS# (Chemical Abstracts Service Registry #) for the chemical. In that case, the controller 108 may retrieve the composition information automatically by searching the PubChem database and/or the Chemical Abstracts Service Registry.

In another example, the CSA system 100 may allow a user to provide the composition information by simply selecting or uploading a safety data sheet (SDS). The controller 108 may analyze the SDS to detect any chemicals and components with a CAS# in the SDS. The controller 108 may use optical character recognition (OCR) to parse the data on the SDS and detect the relevant information. The controller 108 may automatically populate the relevant information detected from the SDS to the input boxes on the GUI 140. Optionally, the user may both manually enter composition information and upload an SDS on the same run. Optionally, the user can upload the SDS or another document that contains composition information for a chemical to be evaluated by scanning or imaging the document via the optical sensor 120. In another example, the user may simply upload the SDS or other document into the CSA system 100 from a webpage or a storage folder on the computer device 102.

The CSA system 100 may prompt the user to provide optional additional inputs, such as weight settings for the sustainability parameters, hazardous waste disposal inputs, a GWP value, material cost, and/or the like. For example, the CSA system 100 permits the user to define and/or adjust the scaling factors that affect the sustainability value 214 that is output. In an example, if the user does not select the weight settings, the controller 108 may proceed with the assessment using default weight settings for the sustainability parameters. Optionally, the default weight settings may be that all sustainability parameters are equally weighted. The optional hazardous waste disposal inputs may be geographical information and/or transport information for each of the constituent components of the chemical. The CSA system 100 may permit the user to manually override the default settings and internal calculations/algorithms. For example, the user may be allowed to manually input the material costs of the constituent components and/or the GWP value, instead of relying on the data that the controller 108 obtains from the various data sources 206.

The calculation steps 208 calculate scores for each of multiple different sustainability parameters 212. For example, the controller 108 may calculate a score for each of the sustainability parameters, and then may scale and aggregate at least some of those parameter scores to generate a sustainability value 214 for the given chemical. The scores for the sustainability parameters are generate based on the composition information of the chemical to be evaluated. The sustainability value 214 represents one of the outputs 210. In an example, the sustainability value 214 is a quantitative numerical value within a set range, such as 0 to 10 or 0 to 100. The sustainability value 214 may indicate how environmentally friendly the chemical is, and/or how a planned use of the chemical may impact the environment, or people using it, based on the sustainability parameters 212 that are considered. As an example, the range may be 0 to 10, with 10 indicating worst and 0 indicating best for the environment (e.g., least negative impact). A first chemical with a sustainability value 214 of 3 would be considered more environmentally friendly than a second chemical with a sustainability value of 7.

In the illustrated example, there are nine sustainability parameters 212 considered by the controller 108 to generate the sustainability value 214. The sustainability parameters 212 include a toxicology parameter 216, a flammability parameter 218, a reactivity parameter 220, a global warming potential (GWP) parameter 222, an ozone depletion parameter 224, a volatile organic compounds (VOC) parameter 226, a material cost parameter 228, a regulatory risk parameter 230, and a hazardous waste parameter 232. The controller 108 may consider additional parameters, fewer parameters, and/or one or more different parameters in other embodiments.

The controller 108 may generate a respective score or metric for each of the sustainability parameters 212 that are considered relevant. To generate the respective scores, the controller 108 may access and search various data sources, including the database sources 206 shown in FIG. 3. The controller 108 may search for classification of a chemical on various regulation/regulatory databases. The controller 108 may also search for a chemical designation as less environmentally hazardous (e.g., less toxic and/or harmful to the environment). The database sources 206 shown in FIG. 3 include PubChem API, Cheminformatics, , VOC Exempt List, Prop 65, the EPA Clean Air Act, Regulatory Databases, and 40 CFR Part 261. In an example, the controller 108 may use the PubChem record to source all primary chemical information, including hazards, structure, and physical properties of the chemical to evaluate during an assessment. The controller 108 may assess GHS hazard codes and/or statements for a TURI P2OASYS calculator. The controller 108 may check a number of regulations and databases for chemical classification to generate the respective score for one or more of the parameters 216, 218, 220, 222, 224, 226, 228, 230, 232. The regulations and databases that are searched by the controller 108 may include, for example, the EPA Clean Air Act; the EPA List of Hazardous Air Pollutants (HAP); the EPA List of Ozone Depleting Substances (ODS) (Title VI of the Clean Air Act); California Prop 65; the Aerospace and Defense Declarable Substances List (AD DSL); the US Toxic Substances Control Act (TSCA); the European Chemicals Agency (ECHA), the Registration, Evaluation, Authorization, and Restriction of Chemicals (REACH); the EPA Per- and Polyfluoroalkyl Substances (PFAS) ToxCast

Chemical Inventory; the EPA Toxics Release Inventory (TRI); the EPA CERCLA Hazardous Substances (40 CFR 203.4); the SARA Section 311/312/313 Hazard; the US EPA Emergency Planning and Community Right-To-Know Act (EPCRA); the SARA Title III (40 CFR 355, and 40 CFR 372.65); the US EPA Resource Conservation and Recovery Act (RCRA) Composite List of Hazardous Wastes (40 CFR 261); State Right-To-Know Substance Lists; the EPA Per- and PolyFluoroalkyl Substances (PFAS) ToxCast Chemical Inventory; the EPA Toxic Release Inventory (TRI); the 40 CFR Part 261 Identification and Listing of Hazardous Waste, AD-RSL; and/or the like.

The controller 108 may be programmed to access some of these databases on every run of the calculation, or on a defined revalidation time interval, for up-to-date listings. For example, the controller 108 may source non-chemical-specific regulatory information daily, and the results may be stored in a database that is accessible to all user accounts. Other databases may have an uploaded version that must be updated in the program when new versions are released. The controller 108 may access the databases via the network 104, such as by communicating with the one or more remote computing systems 106. On the GUI 140, the controller 108 may display the specific regulatory sub-code corresponding to a chemical along with any listed exemptions. The controller 108 may conduct similar database searches for designation of a chemical as less environmentally hazardous or benign (e.g., less toxic and/or harmful to the environment). These databases include, but are not limited to, the EPA Safer Chemical Ingredients List (SCIL).

The toxicology parameter 216 may be a semi-quantitative or quantitative assessment of toxicological and/or ecological hazards associated with the chemical. In an example, the controller 108 may assess toxicology based on one or more databases and/or scoring tools. Example scoring tools include the Toxics Use Reduction Institute (TURI) Pollution Prevention Options Analysis System (P2OASYS) and the Quick Chemical Assessment Tool (QCAT). Example databases relevant to toxicology and GHS hazards include EPA Cheminformatics, Japan National Institute of Technology and Evaluation (NITE), and EPA CompTox. In an embodiment, the controller 108 may cumulatively account for hazards of all components in a mixture at equal weighting regardless of the weight %. For example, the hazards of all constituent components may be equally factored in the toxicology score, regardless of weight settings defined by the user.

In an example, the toxicology parameter 216 may include acute human toxicity, chronic human toxicity, and fish/aquatic invertebrate toxicity endpoints (e.g., sub-sections). These sub-sections may be classified into NFPA Health, inhalation toxicity, oral toxicity, dermal toxicity, respiratory irritation, dermal irritation, eye irritation, chronic organ effects, specific target organ toxicity, respiratory sensitivity/disease, mutagen/teratogen, carcinogen, acute aquatic toxicity, and chronic aquatic toxicity. The controller 108 may assess the GHS hazard statements from the constituent components cumulatively. For example, the controller 108 may assume that the final mixture inherits all hazards from the components regardless of their % in the mixture.

The controller 108 may assess all components for data gaps in toxicological information using the Cheminformatics data hub or ECHA chemicals database and brief profiles. In an embodiment, the controller 108 may employ a conservative approach for dealing with missing information, to ensure that the resulting sustainability value is not artificially characterized as less toxic. The controller 108 may assign the worst score possible (e.g., a score of 10) for any sub-sections identified as inconclusive or with no existing data, so the data gaps will not result in the chemical appearing safer or more sustainable than it actually is. In an embodiment, the controller 108 may perform this conservative scoring for groups of toxicology endpoints (Chronic Human Health, Acute Human Health, and Ecological Toxicity) if data is missing from a sufficient number of contributing categories (i.e., STOT SE/RE) or is missing from categories deemed essential to scoring for that endpoint (e.g., sustainability parameter). For example, this endpoint grouping may be performed according to the GreenScreen method for Safer Chemicals.

The flammability parameter 218 may be a semi-quantitative or quantitative assessment of flammability hazards associated with the chemical. The controller 108 may analyze various data sources 206 to determine data variables 204 related to flammability. The data sources accessed by the controller 108 for flammability may include NFPA fire rating, flash point, vapor pressure, and associated GHS hazard statements. The controller 108 may assume that flammability GHS hazards are cumulative in the final mixture. The controller 108 may choose to specify the physical properties of a mixture input into the CSA system 100 via an SDS upload by sourcing the vapor pressure and flash point directly from the SDS.

The reactivity parameter 220 may be a semi-quantitative or quantitative assessment of reactivity hazards associated with the chemical. The controller 108 may analyze various data sources 206 to determine data variables 204 related to reactivity. The data sources by the controller 108 for reactivity may include the NFPA instability rating, potential for peroxide formation, and associated GHS hazard statements. The controller 108 may assume that reactivity hazards are cumulative in the final mixture.

The GWP parameter 222 may be a semi-quantitative or quantitative assessment of global warming potential of the chemical. The controller 108 may use a 100-year time frame to calculate equivalent carbon emissions, with scores adjusted for predicted atmospheric lifetime. The GWP parameter may be defined by the relative heat trapped in the atmosphere by 1 ton of material/chemical compared to 1 ton of carbon dioxide (CO2). The calculation uses the anticipated degradation and interaction pathways of a chemical based on functional groups in the structure to calculate CO2 equivalents (CO2e) of greenhouse gases (GHGs) produced. The controller 108 may use EPA-published values for hydrofluorocarbons (HFCs), when available, to replace estimates from the anticipated degradation pathway. The calculated GWP may be structure-based. The GWP parameter may only capture GHGs produced at end-of-life for a chemical but does not include industrial synthesis or processing. In cases where industrial emissions are a significant contributor, the user can manually enter a GWP value for the industrial emissions, via a user input device 118, to override the calculation. For chemical mixtures, the controller 108 may calculate the GWP parameter as a scaled average. The controller 108 may weight each constituent component in the calculation by the weight percentage of that component in the mixture. The controller 108 may assume that the components do not react with one another and/or any reaction between components does not modify the GHGs released over product lifetime and end-of-life. The controller 108 may scale the final GWP value using a normalization function, such as a sigmoid, to have adequate differentiation of scores while capturing very high GWP materials. For example, the controller 108 may scale the GWP value so that the score of the GWP parameter 222 is within a defined range (e.g., 0 to 10). In an example, the controller 108 may use sigmoid normalization functions that compress the domain of real numbers into a range of 0,1. The controller 108 may scale the compressed value with a multiplicative factor of 10 to ensure that output values fall within the desired range. The domain over which sensitivity remains high may be controlled by an exponential factor in the denominator (e.g., 10*Sigmoid(x)ϵ[0,10]).

The ozone depletion parameter 224 may be a semi-quantitative or quantitative assessment of ozone depletion potential of the chemical. The controller 108 may determine the ozone depletion score based on the GHS H420 hazard classification. The controller 108 may score the ozone depletion parameter 224 as a binary designation. For example, the controller 108 may determine that the evaluated chemical does contribute to ozone depletion (e.g., yes) or does not contribute to ozone depletion (e.g., no). In multi-component mixtures, the controller 108 may determine the score as the % of ozone- depleting constituent components in the mixture.

The VOC parameter 226 may be a semi-quantitative or quantitative assessment of volatile organic compounds in the chemical. The controller 108 may use the boiling point of each component in a mixture to designate the component as a VOC or not a VOC, based on an EPA classification of VOCs as chemicals with boiling points below 240 °C. The controller 108 provides a binary yes or no score for pure compounds. For mixtures, the controller 108 may determine whether each of the components is a VOC or not, and then calculate the % of VOCs in the mixture. The controller 108 may search the VOC Exempt Database for exceptions, which will be given a score of 0 regardless of boiling point. The VOC Exempt List refers to a list of chemicals that are not considered to be VOCs and are exempt from regulations that restrict the use of VOCs. Chemicals that are VOC exempt do not meaningfully contribute to environmental emissions. Vapor pressure is also used to check if components may meet requirements to potentially become VOC exempt, but this designation does not affect the score.

The cost parameter 228 may be a semi-quantitative or quantitative assessment of the chemical cost to use the chemical for the intended use application. The controller 108 may estimate bulk pricing for the chemical that is assessed by searching chemical suppliers for small-volume pricing and dividing by a scale-up factor. The controller 108 may search for small volume pricing from common suppliers. In an example, the controller 108 may assumes a decrease in price for bulk orders, such as a 10X decrease). Optionally, the user can manually input [bulk] pricing via an input device 118, if available and/or desired by the user. The manual input overrides the pricing data obtained by searching for supplier pricing. The controller 108 may input a reference cost value and scale the final cost value using a sigmoid function so that the score is within a designated (e.g., 0 to 10) range centered on the reference value. For example, the controller 108 may input a reference value for an incumbent material to assess the relative cost with representative differentiation of scores even for very high-cost materials.

The regulatory risk parameter 230 may be a semi-quantitative or quantitative assessment of regulatory risk associated with the chemical. The regulatory risk parameter 230 score indicates if using the chemical would violate or be subject to restrictions by an environmental or toxicological regulation. In one example, when receiving user inputs 202 related to the regulatory risk parameter 230, a de minimus exemption 229 can be applied to the user inputs to prevent consideration as a regulatory risk parameter 230. The controller 108 may calculate an average of relative scores for identified listing or classification on all included regulatory databases (both positive, e.g.

SCIL, and negative, e.g. AD-DSL). Regulated chemical classes may be identified by collective structural motifs and/or elemental fractions. In some cases, a chemical class may be indicated but not confirmed by identified structural motifs and the user may be prompted to confirm or reject the class identification. This may be applicable when a regulation includes classes like short chain chlorinated paraffins (SCCPs), which are not a discrete chemical but rather a group of chemicals with common structural components. The controller 108 attempts to identify these components and, when possible, identify the class or prompt the user in relevant cases to confirm/deny if the chemical is a part of this class. The controller 108 may also prompt the user to confirm or deny based on an intended use application for the chemical. When relevant, the controller 108 may score classifications for degree or type of restriction according to impact. Exemptions are identified and may modify the regulatory risk score.

The hazardous waste parameter 232 may be a semi-quantitative or quantitative assessment of hazardous waste classification and/or hazardous waste disposal by using the chemical for an intended use application. The controller 108 may first identify a chemical waste type generated by the chemical. The controller 108 may access the 40 CFR Part 261 Identification and Listing of Hazardous Waste to assign the Resource Conservation and Recovery Act (RCRA) standardized waste type. These are F for non-specific source waste, K for specific source waste, U for hazardous commercial chemicals, and P for acutely hazardous chemicals, with sub-classifications according to chemical composition or properties. The controller 108 may assign a high hazardous waste score in response to identifying that the chemical is in a high hazardous waste class.

For cases including hazardous waste disposal, the same base scoring for hazardous waste type may be completed and scored along with a disposal difficulty score. This sub-score employs user input for geographic and/or transport information to assess hazardous waste disposal factors including but not limited to emissions from transport to disposal site, emissions from waste treatment, required level of waste treatment, and associated cost.

After the scores for the various sustainability parameters are determined, the controller 108 scales the scores according to user-definable weight settings at the scaling step 234 and generates the sustainability value 214 based on the scaled scores of the sustainability parameters. The CSA system 100 in an embodiment allows the user to dynamically control/adjust the weight setting of each sustainability parameter that is factored into the output sustainability value 214. For example, the user has the ability to adjust the weight settings of one or more of the sustainability parameters based on perceived importance of the sustainability parameters with respect to an intended use application for the chemical. Sustainability parameters that the user determines are not of interest may be diminished or even completed excluded from the calculation. The user may adjust the weight settings by manipulating a touch-based input device 118 to move or toggle graphic indicia displayed on the GUI 140. For example, the GUI 140 may display slider bars next to each of the sustainability parameters. The user may control an input device 118 to move the slider bar for a given parameter in one direction to increase the weight of that parameter in the output sustainability value 214, and may move the slide bar in the opposite direction to decrease the weight of the parameter. The user may decrease the weight of the parameter to zero to exclude that parameter from the sustainability value calculation. After adjusting the weight settings, the controller 108 generates the sustainability value 214 based only on the scores and the current weight settings of the sustainability parameters that remain.

In an example, the CSA system 100 may be used in an attempt to find a replacement chemical for a Halon compound in a product. The user has one or more candidate chemicals to evaluate using the CSA system 100. The user may determine that GWP and hazardous waste are important considerations. Therefore, the user may increase the weight settings for these two highly relevant sustainability parameters so that the scores for the GWP parameter 222 and the hazardous waste parameter 232 have a greater influence on the resulting sustainability value 214 that is output. For example, the user may set the weight settings for these two parameters to a maximum available level (e.g., 10, 100%, etc.). The user may remove other parameters from the analysis or set the weight settings for one or more other parameters to a lower level than the weight settings for the highly relevant parameters. In another example, the user’s main concern may be cost profile and safety of the chemical during prolonged human exposure (e.g., the chemical is used in a confined space). In that scenario, the user may increase the weight settings of parameters such as toxicology 216 (e.g., toxic use reduction), cost 228, and flammability 218.

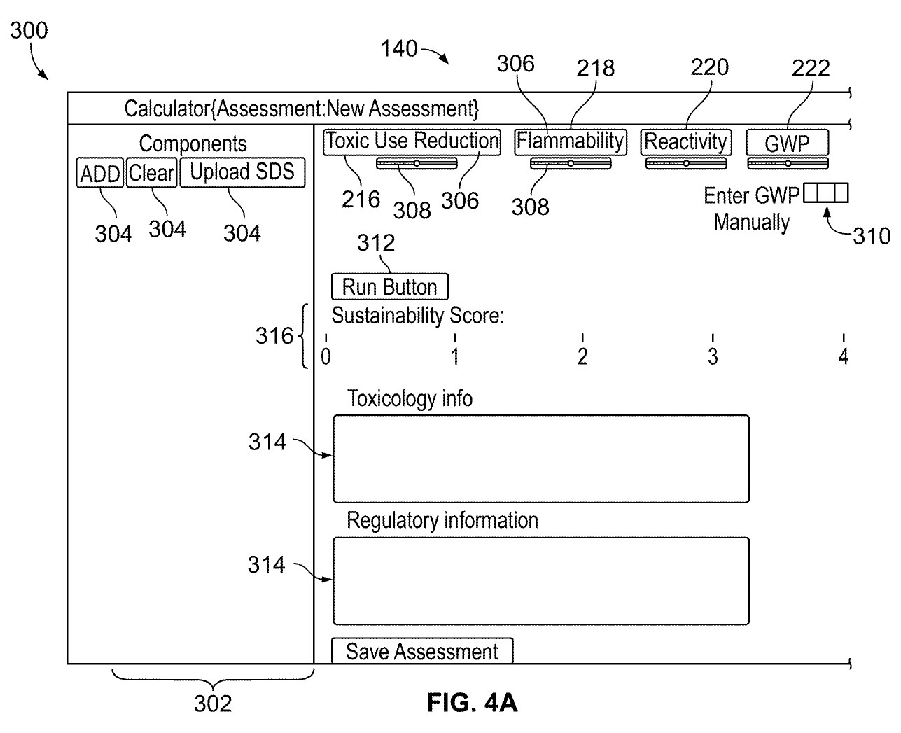

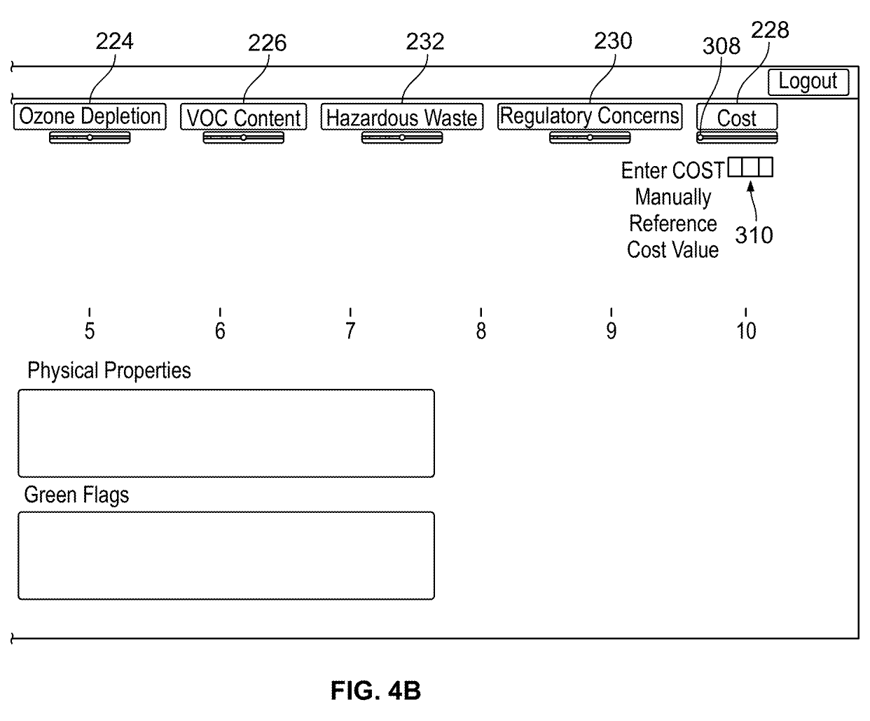

FIG. 4 shows an example assessment setup page 300 on the GUI 140 of the CSA system 100 according to an embodiment. The assessment setup page 300 may include information, input boxes, and user-selectable elements that permit the user to set up and run a new assessment of a chemical. The left sidebar 302 provides the composition of the chemical to be evaluated. No composition is currently shown, but there are user-selectable elements 304 that enable the user to “add” a new composition, “clear” an existing composition, or “upload [an] SDS”. The top of the page 300 shows user-selectable elements 306 for each of the sustainability parameters 216, 218, 220, 222, 224, 226, 228, 230, 232 and associated user-adjustable slider bars 308 for the weight settings. The slider bars 308 of all of the sustainability parameters are equal in FIG. 4, except for the cost parameter 228. For example, the weight settings of most of the sustainability parameters are 50%, and the weight setting for cost is 0. This means that the cost parameter 228, according to the current settings, would be omitted from factoring into the sustainability value 214 that is output. The user can adjust the weight setting up or down, for each parameter independently of the other parameters, by dragging the corresponding slider bar 308.

As described above, the CSA system 100 may provide the opportunity for the user to manually input scores (e.g., metrics) for the GWP parameter 222 and the cost parameter 228. The assessment setup page 300 of the GUI 140 may include input boxes 310 in which the user can manually enter data related to GWP and/or cost. For example, if an SDS upload indicates that at least 50% of the chemical composition is unknown and/or confidential (e.g., protected by trade secret), the controller 108 may automatically deactivate (e.g., set the weight settings to zero) one or more of the sustainability parameters to maintain calculation accuracy. For example, the controller 108 may automatically deactivate the GWP parameter 222, the VOC content parameter 226, and the material (e.g., chemical) cost parameter 228. This prevents improper assessment of mixtures without sufficient information. Other sustainability parameters can still be calculated accurately using the hazard statements in the SDS(s). To override the deactivation, the user may manually enter a value for global warming potential or cost if known.

The assessment setup page 300 may include a user-selectable element 312 that initiates the controller 108 to generate the sustainability value 214. In the illustrated example, the user-selectable element 312 is a virtual button labeled “Run Button,” although the element 312 may have other names. Once the user is finished setting up the assessment, the user can select the button 312 via a user input device 118. Selecting the button 312 initiates the controller 108 to perform the assessment for the identified chemical. Below the Run button 312 may be information windows or boxes 314, a scale 316, and/or the like that are used to display the results of the assessment. In FIG. 4, these boxes and the scale are not populated with information until the assessment is performed.

The CSA system 100 may generate pop-up notifications that present regulatory information and/or data acquisition warnings. The controller 108 may display the pop-up notifications when the user selects the chemical to assess and/or in response to the user selecting the Run button 312 to initiate the assessment. The user can close these pop-up notifications to view the results of the assessment.

The controller 108 generates the sustainability value 214 based on all of the includes sustainability parameters and the respective weight settings. For example, the controller 108 may input the scores of the sustainability parameters and the user-scaled weight settings for the sustainability parameters into an algorithm, function, mathematical model, or the like, that calculates the sustainability value 214 as the output of the algorithm or function. The controller 108 generates the sustainability value 214 for the assessed chemical so that the scores of the parameters that have greater weight settings have greater affect or impact on the output value than the scores of the parameters that have lower weight settings.

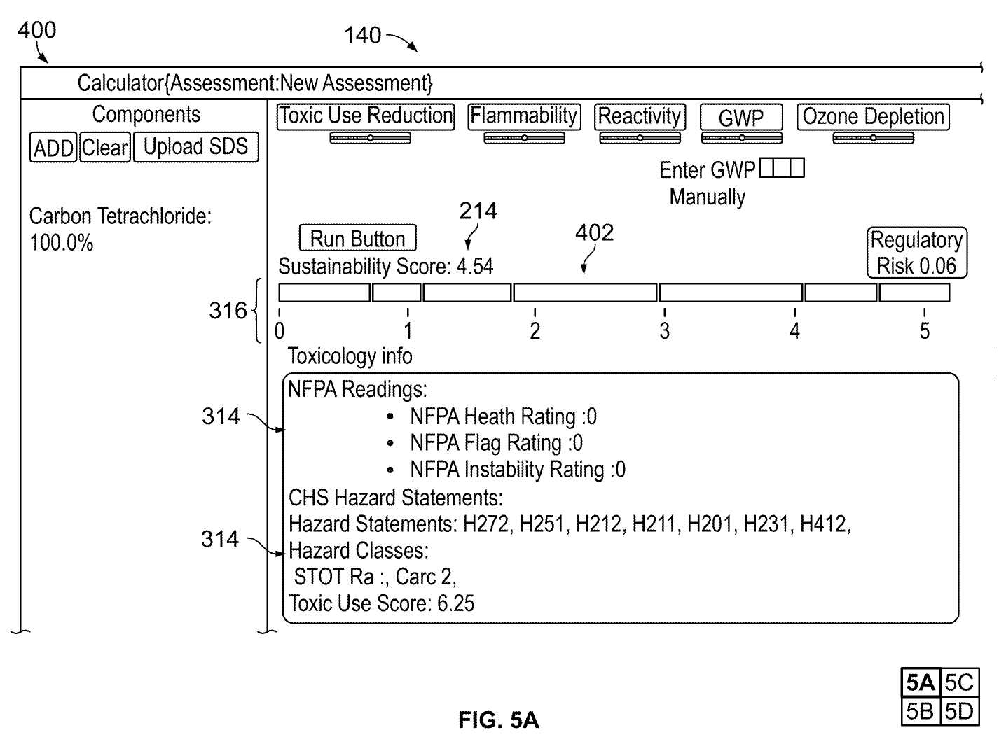

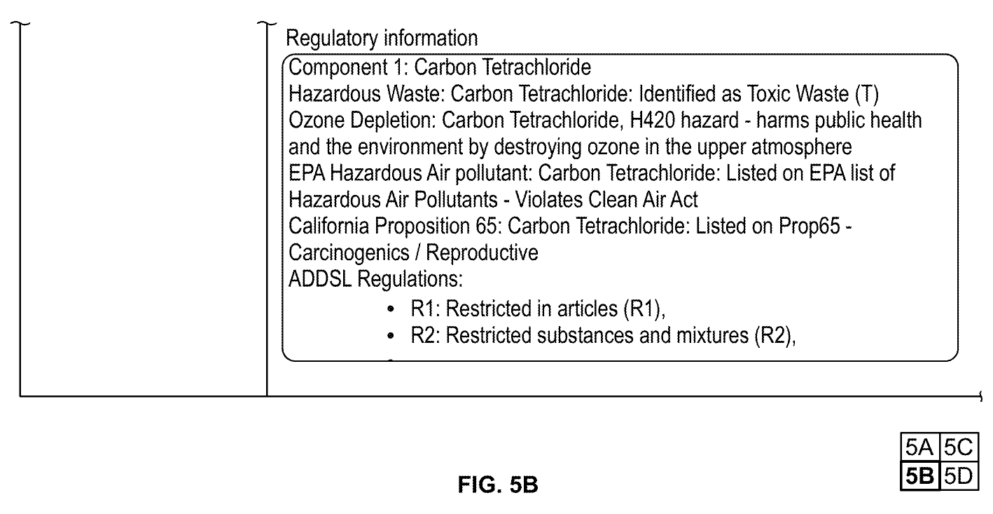

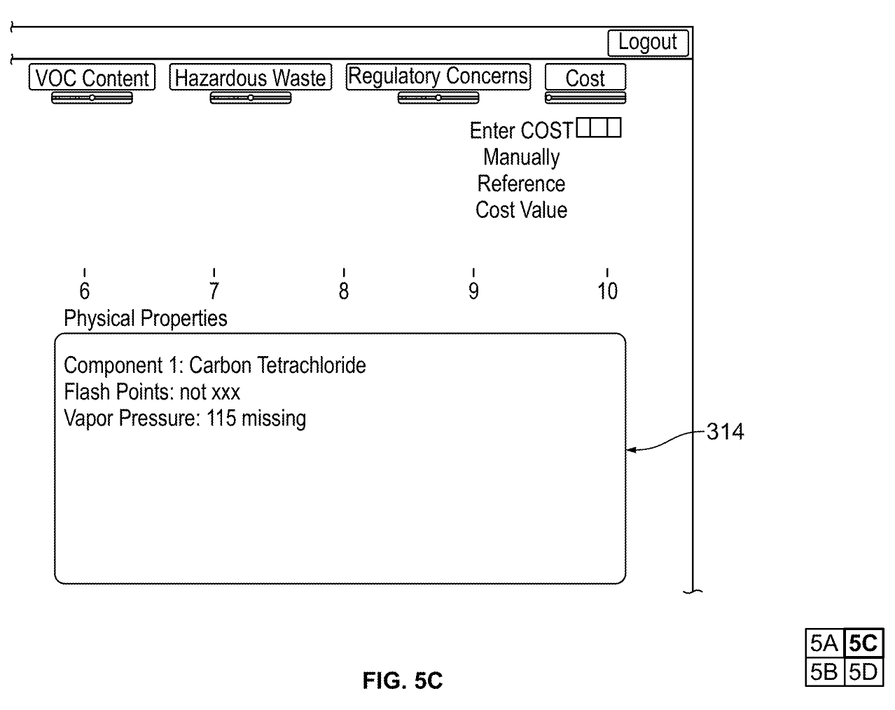

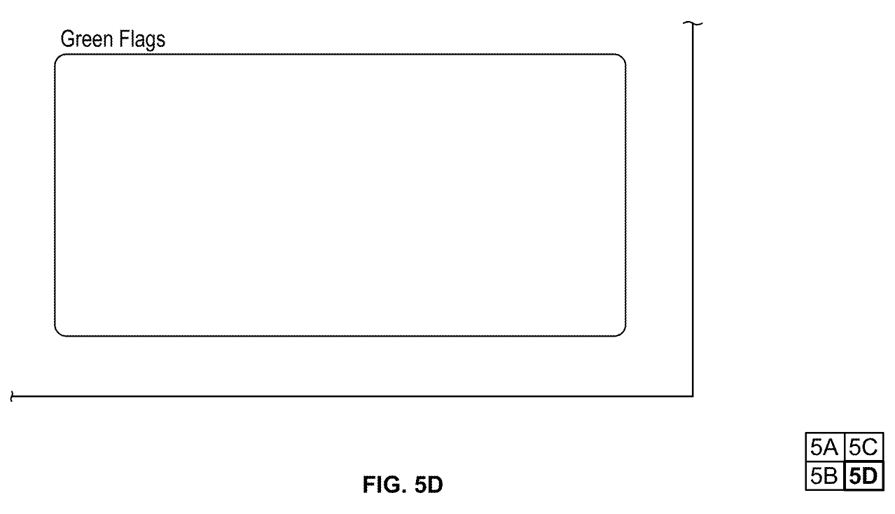

FIGS. 5A-5C show an example assessment result page 400 on the GUI 140 of the CSA system 100 according to an embodiment. In the illustrated example, the chemical that is assessed is pure Carbon Tetrachloride (e.g., 100 wt.%). The GUI 140 presents the sustainability value 214 for Carbon Tetrachloride as 4.54. For example, the controller 108 may generate the sustainability value 214 as a quantitative, numerical value, which enables easy comparison between chemicals. In addition to presenting the final value 214, the controller 108 may generate the GUI 140 to present a graphical breakdown 402 of each of the relevant sustainability factors that impacted the sustainability value 214. The graphical breakdown 402 is shown as segmented bar on the scale 316. This allows the user to visualize the sustainability parameters that contributed to the output value 214. In an example, a lower score indicates a more sustainable, lower risk, and/or less hazardous chemical than another chemical that has a higher score. The illustrated value of 4.54 may be on a scale 316 from 0 to 10.

In an example, the controller 108 may control the GUI 140 to display additional information in response to the user’s cursor hovering over or selecting active user interface elements. For example, the user can move the cursor to hover over different segments of the segmented bar graphical breakdown 402. In FIG. 5, the controller 108 displays the score of the regulatory risk parameter 230 in response to a cursor either selecting or hovering over the distal segment of the segmented bar 402 which is associated with regulatory risk. The regulatory risk score in the illustrated example is 0.56. The additional information may be displayed in pop-up windows that are removed in response to the cursor moving away from the active user interface element. The user can move the cursor to different segments of the bar 402 to view additional information about different sustainability components that impacted the sustainability value 214.

The GUI 140 may also display relevant regulatory information and hazard information associated with the chemical that is evaluated. This information may be displayed in the information windows or boxes 314 below the scale 316. Example output information 210 (shown in FIG. 3) that may be presented can include relevant GHS hazard codes, GHS hazard descriptions, green flags, physical properties, and/or regulatory concerns for the chemical. The GHS hazard codes and descriptions are part of the hazardous waste parameter 232. The GHS hazard codes may also factor in other parameters such as flammability 218, reactivity 220, and toxicology 216.

The green flags indicate that a chemical has been deemed by a regulatory listing (e.g., the EPA) as a relatively safe chemical choice. Another green flag may be that a chemical is listed on the VOC exempt list. If the assessed chemical is VOC exempt, then the controller 108 may not provide any score for the VOC parameter 226, but may notify the user that the chemical is VOC exempt. Even if the chemical has one or more green flags, the controller 108 may still calculate a score for the toxicology parameter 216. For example, the SCIL classifications are use-case dependent. The CSA system 100 may not assume the user’s use case, but may be configured to provide helpful information suggestive of use applications. For example, the controller 108 may present in one of the boxes 314 that the chemical could be used as a “cleaner solvent.” In the illustrated embodiment, regulatory listings are shown in the bottom left box 314, green flags are described in the bottom right box 314, physical properties are shown in the top right box 314, and toxicology information is shown in the top left box 314.

In an embodiment, the controller 108 may save performed assessments in a storage device, such as the memory 112 of the computing device 102 or a memory device of a remote computing device 106. The assessments may be stored to enable future comparisons between different chemicals. For example, the controller 108 may store all details about the assessment, including the composition information of the chemical, the scores and weight settings of the sustainability parameters, and the sustainability value 214. The saved file may also include all regulatory and hazard information relevant to the assessment. For example, the controller 108 may store hazard information for each component in the chemical, regulatory concerns by component, and green flags by component. On subsequent runs of the CSA system 100, the stored data may be uploaded to automatically set the weight settings to those previously used.

Optionally, the controller 108 may generate the GUI 140 to display a list of completed assessments in the left sidebar 302. The user is able to select the chemicals from the list to compare the chemicals or simply revisit the details of the completed assessments.

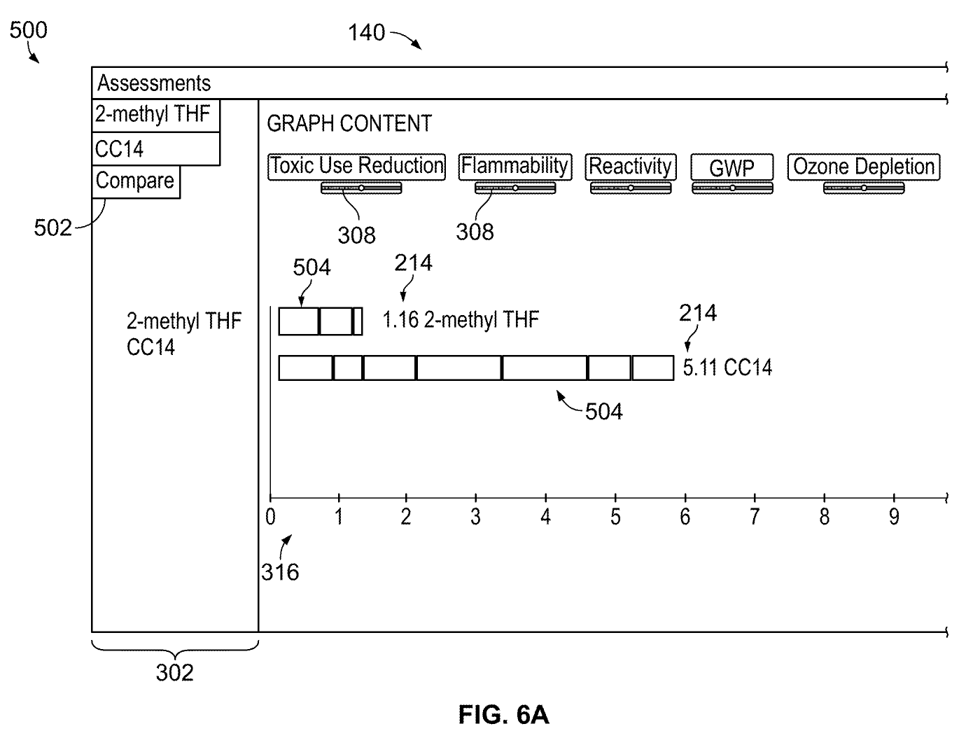

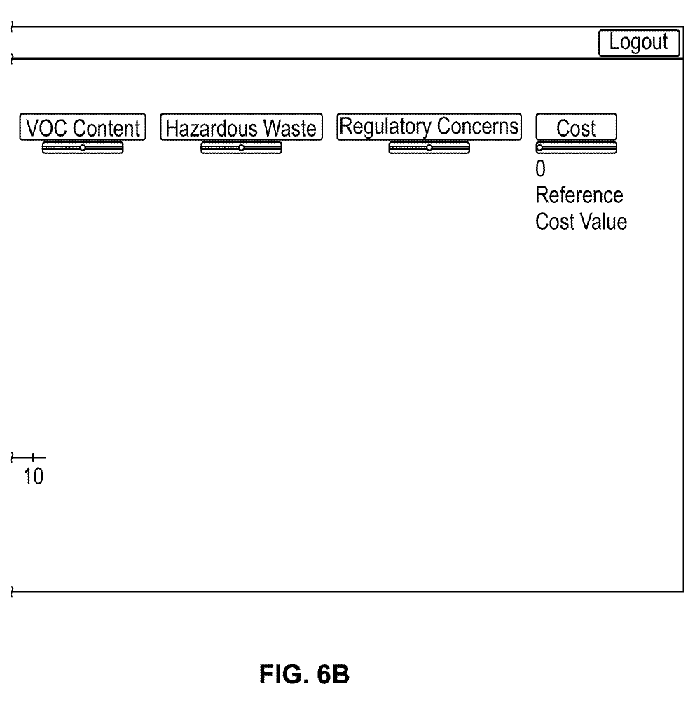

The CSA system 100 can perform side-by-side comparisons of multiple chemicals. The user can set up a comparison after multiple assessments have been performed and saved. For example, a user can set up a comparison by selecting two chemicals listed in the left sidebar 302 (shown in FIG. 4) and then selecting a user interface element associated with a comparison function. FIG. 6 shows an example comparison page 500 on the GUI 140 of the CSA system 100 according to an embodiment. The left sidebar 302 shows two chemicals listed including 2-methyl THF and CCl4. There is a user interface element 502 that is a virtual “Compare” button below the two listed chemicals. The user may select the chemicals from the list that are desired for comparison, and then actuate the Compare button 502 to initiate a comparison between the two selected chemicals. Optionally, the CSA system 100 may enable comparing more than two chemicals at the same time.

Because the weight settings for the sustainability parameters are adjustable, the weight settings that were used to perform the initial assessments of the two chemicals may not match. In an embodiment, the controller 108 automatically adjusts the weight settings for the sustainability parameters so that common weight settings are used in the comparison. For example, the controller 108 may use the same weight settings to generate the sustainability values for the two chemicals in the comparison. As a result, the weight settings for at least one of the chemicals in the comparison may be different than the weight settings used in the previous assessment for the same chemical, causing the sustainability value for that chemical to change in the comparison relative to the stored previously-performed assessment. In an embodiment, the CSA system 100 may still permit the user to adjust or scale the weight settings as desired. For example, the user can adjust one or more of the slider bars 308 as desired to modify the weight settings. In this case, the user-provided changes are uniformly applied to both compared chemicals. For example, the slider bars 308 control the weight settings for the sustainability parameters that are applicable to all of the chemicals in the comparison. Once the user is satisfied with the weight settings and the setup, the user can select the Compare button 502 to run the comparison assessment. The controller 108 may reassess each of the chemicals if any weight settings or other factors have been changed since the previous assessment of that chemical.

In the illustrated example, the 2-methyl tetrahydrofuran (2-methyl THF) chemical has a sustainability value 214 of 1.15 and the CCl4 chemical has a sustainability value 214 of 5.11. This example indicates that the 2-methyl THF chemical is more sustainable than the CCl4 chemical. The 2-methyl THF chemical would be the preferred choice in this example due to the anticipated environmental impacts and affect on human and animal health. The controller 108 may display a side-by-side graphic comparison of the sustainability values 214 as segmented bars 504 along a common scale 316. The controller 108 may also display relevant GHS Hazard codes, GHS Hazard descriptions, green flags, physical properties, and/or regulatory concerns for the compared chemicals. This additional information may be presented in the boxes 314 (shown in FIGS. 4 and 5).

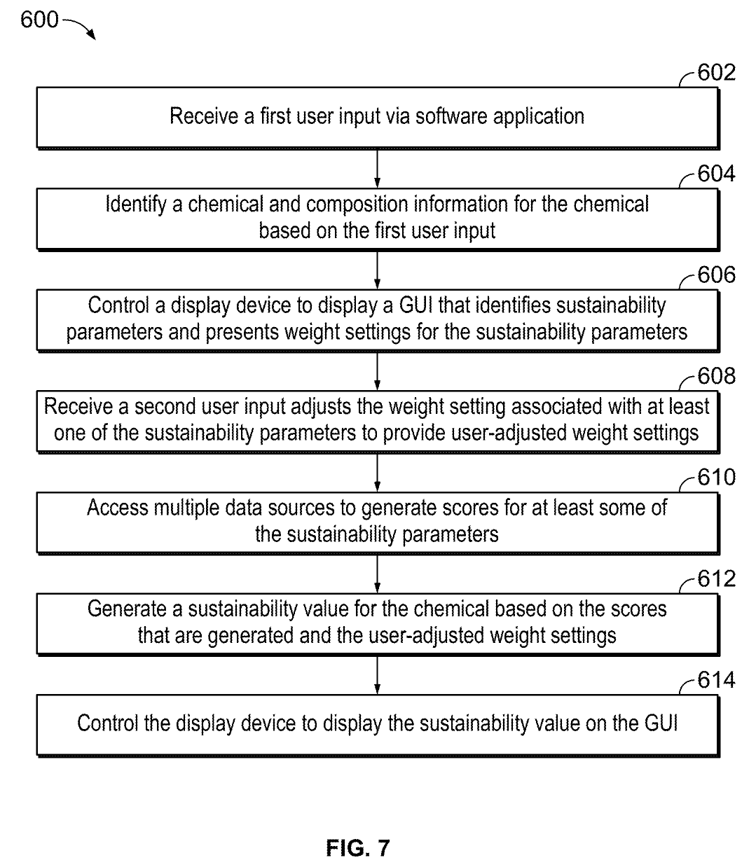

FIG. 7 is a flow chart 600 of a method for performing a chemical sustainability assessment according to one or more embodiments. The method is a computer-implemented process that uses one or more non-transitory, computer-readable memory devices and one or more processors of a computing device operably connected to the memory device(s). The method may be performed, at least in part, by the controller 108 (e.g., the processor(s) 110) of the CSA system 100. The memory devices may include the memory 112 and/or the remote computing device(s) 106, such as at least one server. The method may include additional steps than shown in FIG. 7, fewer steps than shown in FIG. 7, a different order of the steps, and/or different steps than shown in FIG. 7.

With additional reference to FIGS. 1-6, the method begins at step 602, at which a first input is received from a user via a software application 146 operating on a computing device 102. At step 604, a chemical and composition information for the chemical are identified by the controller 108 based on the first input. At step 606, the controller 108 controls a display device 116 to display a graphical user interface (GUI) 140 that identifies sustainability parameters and presents weight settings for the sustainability parameters.

At step 608, the controller 108 receives a second input from the user via the software application 146. The second input adjusts the weight setting associated with at least one of the sustainability parameters to provide user-adjusted weight settings. At step 610, the controller 108 accesses multiple data sources to generate scores for at least some of the sustainability parameters. At step 612, the controller 108 generates a sustainability value 214 for the chemical based on the scores that are generated and the user-adjusted weight settings. At step 614, the controller controls the display device 116 to display the sustainability value 214 for the chemical on the GUI 140.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

It is to be understood that the above description is intended to be illustrative, and not restrictive. For example, the above-described embodiments (and/or aspects thereof) can be used in combination with each other. In addition, many modifications can be made to adapt a particular situation or material to the teachings of the various embodiments of the disclosure without departing from their scope. While the dimensions and types of materials described herein are intended to define the parameters of the various embodiments of the disclosure, the embodiments are by no means limiting and are exemplary embodiments. Many other embodiments will be apparent to those of skill in the art upon reviewing the above description. The scope of the various embodiments of the disclosure should, therefore, be determined with reference to the appended claims, along with the full scope of equivalents to which such claims are entitled. In the appended claims and the detailed description herein, the terms "including" and "in which" are used as the plain-English equivalents of the respective terms "comprising" and "wherein." Moreover, the terms "first," "second," and "third," etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the various embodiments of the disclosure, including the best mode, and also to enable any person skilled in the art to practice the various embodiments of the disclosure, including making and using any devices or systems and performing any incorporated methods. The patentable scope of the various embodiments of the disclosure is defined by the claims, and can include other examples that occur to those skilled in the art. Such other examples are intended to be within the scope of the claims if the examples have structural elements that do not differ from the literal language of the claims, or if the examples include equivalent structural elements with insubstantial differences from the literal language of the claims.

What is claimed is:

1. A computer-implemented method for performing a chemical sustainability assessment, the method comprising:
receiving a first input from a user via a software application operating on a computing device;
identifying, via one or more processors of the computing device, a chemical and composition information for the chemical based on the first input;
controlling a display device to display a graphical user interface (GUI) that identifies sustainability parameters and presents weight settings for the sustainability parameters;
receiving a second input from the user via the software application, the second input adjusting the weight setting associated with at least one of the sustainability parameters to provide user-adjusted weight settings;
accessing multiple data sources to generate scores for at least some of the sustainability parameters;
generating a sustainability value for the chemical based on the scores that are generated and the user-adjusted weight settings; and
controlling the display device to display the sustainability value for the chemical on the GUI.

2. The method of claim 1, wherein the chemical is a first chemical and the sustainability value is a first sustainability value; wherein the method further comprises:
receiving a third input from the user selecting the first chemical and a second chemical for a side-by-side comparison; and
controlling the display device to concurrently display both the first sustainability value and a second sustainability value associated with the second chemical on the GUI.

3. The method of claim 2, further comprising automatically adjusting one or more of the weight settings for the sustainability parameters in response to receiving the third input so that the first sustainability value and the second sustainability value are based on common weight settings.

4. The method of claim 1, wherein receiving the first input comprises receiving a safety data sheet (SDS) for the chemical.

5. The method of claim 4, wherein identifying the chemical based on the first input comprises performing optical character recognition (OCR) on the SDS to identify the chemical based on printed text on the SDS.

6. The method of claim 1, wherein identifying the composition information comprises identifying constituent components that make up the chemical and relative proportions of the constituent components in the chemical.

7. The method of claim 1, wherein accessing the multiple data sources to generate scores for at least some of the sustainability parameters comprises generating scores for sustainability parameters including at least two of a global warming potential (GWP) parameter, a volatile organic compound (VOC) content parameter, or a material cost parameter.

8. The method of claim 1, wherein the sustainability parameters include at least five of: (i) a toxicology parameter, (ii) a reactivity parameter, (iii) a flammability parameter, (iv) a global warming potential (GWP) parameter, (v) a volatile organic compound (VOC) content parameter, (vi) an ozone depletion parameter, (vii) a regulatory risk parameter, (viii) a hazardous waste parameter, and (ix) a material cost parameter.

9. The method of claim 1, wherein generating the sustainability value for the chemical comprises providing the sustainability value as a quantitative numerical value within a designated range.

10. The method of claim 1, wherein controlling the display device to display the GUI that identifies the sustainability parameters and presents the weight settings comprises displaying adjustable slider bars on the GUI corresponding to each of the sustainability parameters, and wherein receiving the second input from the user comprises receiving a signal from a user input device indicating that the user is attempting to move at least one of the adjustable slider bars to adjust at least one of the weight settings.

11. The method of claim 1, wherein, responsive to the second input from the user moving the weight setting for a first sustainability parameter of the sustainability parameters to a lowest level, the sustainability value for the chemical is generated without using the first sustainability parameter.

12. The method of claim 1, further comprising displaying at least one of regulatory information relating to the chemical, hazard information relating to the chemical, or a green flag relating to the chemical on the GUI concurrently with the sustainability value so that the sustainability value is not based on the first sustainability parameter.

13. A system for performing a chemical sustainability assessment, the system comprising:
one or more memory devices that are non-transitory and computer-readable; and
one or more processors of a computing device operably connected to the one or more memory devices, the one or more processors configured to:
receive a first input from a user via a software application operating on the computing device;
identify a chemical and composition information for the chemical based on the first input;
control a display device to display a graphical user interface (GUI) that identifies sustainability parameters and presents weight settings for the sustainability parameters;
receive a second input from the user via the software application, the second input adjusting the weight setting associated with at least one of the sustainability parameters to provide user-adjusted weight settings;
access multiple data sources to generate scores for at least some of the sustainability parameters;
generate a sustainability value for the chemical based on the scores that are generated and the user-adjusted weight settings; and
control the display device to display the sustainability value for the chemical on the GUI.

14. The system of claim 13, wherein the chemical is a first chemical and the sustainability value is a first sustainability value, wherein the one or more processors are configured to:
receive a third input from the user selecting the first chemical and a second chemical for a side-by-side comparison; and
control the display device to concurrently display both the first sustainability value and a second sustainability value associated with the second chemical on the GUI.

15. The system of claim 14, wherein the one or more processors are configured to automatically adjust one or more of the weight settings for the sustainability parameters in response to receiving the third input so that the first sustainability value and the second sustainability value are based on common weight settings.

16. The system of claim 13, wherein the one or more processors are configured to receive the first input by obtaining a safety data sheet (SDS) for the chemical selected by the user, and the one or more processors are configured to perform optical character recognition (OCR) on the SDS to identify the chemical based on printed text on the SDS.

17. The system of claim 13, wherein the one or more processors are configured to access the multiple data sources to generate scores for at least some of the sustainability parameters by generating scores for sustainability parameters including at least two of a global warming potential (GWP) parameter, a volatile organic compound (VOC) content parameter, or a material cost parameter.

18. The system of claim 13, wherein the one or more processors are configured to generate scores for at least five of the following sustainability parameters: (i) a toxicology parameter, (ii) a reactivity parameter, (iii) a flammability parameter, (iv) a global warming potential (GWP) parameter, (v) a volatile organic compound (VOC) content parameter, (vi) an ozone depletion parameter, (vii) a regulatory risk parameter, (viii) a hazardous waste parameter, and (ix) a material cost parameter.

19. The system of claim 13, wherein the one or more processors are configured to control the display device to display adjustable slider bars on the GUI corresponding to each of the sustainability parameters, wherein the one or more processors are configured to receive the second input from the user by receiving a signal from a user input device indicating that the user is attempting to move at least one of the adjustable slider bars to adjust at least one of the weight settings.

20. The system of claim 13, wherein in response to the second input from the user moving the weight setting for a first sustainability parameter of the sustainability parameters to a lowest level, the one or more processors are configured to generate the sustainability value for the chemical without using the first sustainability parameter so that the sustainability value is not based on the first sustainability parameter.
